(19) United States
(12) Patent Application Publication
(10) Pub. No.: US 2004/0259899 A1
(43) Pub. Date: Dec. 23, 2004

(54) Combination therapy for constipation

(76) Inventors: Suketu P. Sanghvi, Kendall Park, NJ (US); Thomas A. Boyd, Grandview, NY (US); Paul J. Maddon, Scarsdale, NY (US)

Correspondence Address: Edward R. Gates, Wolf, Greenfield & Sacks, P.C., 600 Atlantic Avenue, Boston, MA, 02210, US

(21) Appl. No.: 10/821,809

(22) Filed: Apr. 8, 2004

Related U.S. Application Data
(60) Provisional application No. 60/461,585, filed on Apr. 8, 2003.

(51) Int. Cl. A61K 31/485

(52) U.S. Cl. 514/282

(57) ABSTRACT

Methods for treating constipation are provided. The methods include administration of peripheral opioid antagonists in combination with laxatives and/or stool softeners. Patients treatable by the invention include those refractory to conventional laxative and stool softener therapy.

### FIELD OF THE INVENTION

The invention relates to combination therapy for treating constipation in patients who are refractory to laxative and stool softener treatment.

### BACKGROUND OF THE INVENTION

Laxatives and stool softeners are well known for the treatment of constipation. These treatments, however, often fail to produce the desired medical outcome, either in the first instance or over time (even when initially helpful). Therefore, a great many subjects with constipation become refractory to laxative and/or stool softener treatments.

Peripheral opioid antagonists have been used to counteract the side-effects of opioid administration for chronic opioid users (e.g., methadone maintenance patients) and for other patients receiving opioids, for example, for pain. One side-effect of exogenous opioid use is constipation, and peripheral opioid antagonists are being tested for relieving such side-effects.

Peripheral opioid antagonists have also been proposed for counteracting gastrointestinal immotility caused at least in part by endogenous opioids, such as ileus, which often occurs after surgical procedures.

The exact cause of constipation in all these settings remains uncertain. It remains controversial whether central nervous system activity plays a significant, if not dominant role. It is uncertain to what extent gastric emptying delays contribute to constipation. It thus remains uncertain whether multiple pathways contribute to constipation and whether an approach affecting a single pathway will be adequate to address constipation in various settings.

To date, investigators have tried to address constipation using opioid antagonists only after discontinuing other therapies which have failed.

### SUMMARY OF THE INVENTION

It is believed that the combination therapy of peripheral opioid antagonists together with laxative or stool softener therapy will yield unexpected, surprising and synergistic improvements in the treatment of constipation. It is believed particularly that laxative and/or stool softener therapy should be administered concurrently with opioid antagonist treatment and that this will have particularly good results in patients who are refractory to laxative and stool softener treatment.

According to one aspect of the invention, a method is provided for treating constipation. The method involves administering to a patient in need of such treatment a laxative and a peripheral opioid antagonist in amounts effective to treat the constipation. The patient can be refractory to laxative therapy. The method can further involve administering an opioid to the patient. In one important embodiment, the opioid is administered chronically. In another important embodiment, the opioid is morphine. In other embodiments, the opioid is selected from alfentanil, anileridine, asimadoline, bremazocine, burprenorphine, butorphanol, codeine, dezocine, diacetylmorphine (heroin), dihydrocodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine-6-glucoronide, nalbuphine, nalorphine, opium, oxycodone, oxymorphone, pentazocine, propiram, propoxyphene, remifentanyl, sufentanil, tilidine, trimebutine, and tramadol. In still other important embodiments, the peripheral opioid antagonist and laxative are administered in either the same or in different formulation(s).

According to another aspect of the invention, a method is provided for treating constipation. The method involves administering to a patient in need of such treatment a stool softener and a peripheral opioid antagonist in amounts effective to treat the constipation. The patient can be refractory to stool softener therapy. The method can further involve administering an opioid to the patient. In one important embodiment, the opioid is administered chronically. In another important embodiment, the opioid is morphine. In other embodiments, the opioid is selected from alfentanil, anileridine, asimadoline, bremazocine, burprenorphine, butorphanol, codeine, dezocine, diacetylmorphine (heroin), dihydrocodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine-6-glucoronide, nalbuphine, nalorphine, opium, oxycodone, oxymorphone, pentazocine, propiram, propoxyphene, remifentanyl, sufentanil, tilidine, trimebutine, and tramadol. In still other important embodiments, the peripheral opioid antagonist and stool softener are administered in either the same or in different formulation(s).

According to one aspect of the invention, a method is provided for treating a patient with a condition calling for laxative or stool softening therapy, including, but not limited to, gastrointestinal immotility. The method involves administering to a patient in need of such treatment a laxative and a peripheral opioid antagonist in amounts effective to treat the condition. Such conditions are described in greater detail below, as is recited herein. The patient can be refractory to laxative therapy. The method can further involve administering an opioid to the patient. In one important embodiment, the opioid is administered chronically. In another important embodiment, the opioid is morphine. In other embodiments, the opioid is selected from alfentanil, anileridine, asimadoline, bremazocine, burprenorphine, butorphanol, codeine, dezocine, diacetylmorphine (heroin), dihydrocodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine-6-glucoronide, nalbuphine, nalorphine, opium, oxycodone, oxymorphone, pentazocine, propiram, propoxyphene, remifentanyl, sufentanil, tilidine, trimebutine, and tramadol. In still other important embodiments, the peripheral opioid antagonist and laxative are administered in either the same or in different formulation(s).

According to another aspect of the invention, a method is provided for treating a patient with a condition calling for laxative or stool softening therapy, including, but not limited to, gastrointestinal immotility. The method involves administering to a patient in need of such treatment a stool softener and a peripheral opioid antagonist in amounts effective to treat the condition. The patient can be refractory to stool softener therapy. The method can further involve administering an opioid to the patient. In one important embodiment, the opioid is administered chronically. In another important embodiment, the opioid is morphine. In other embodiments, the opioid is selected from alfentanil, anileridine, asimadoline, bremazocine, burprenorphine, butorphanol, codeine, dezocine, diacetylmorphine (heroin), dihydrocodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine-6-glucoronide, nalbuphine, nalorphine, opium, oxycodone, oxymorphone, pentazocine, propiram, propoxyphene, remifentanyl, sufentanil, tilidine, trimebutine, and tramadol. In still other important embodiments, the peripheral opioid antagonist and stool softener are administered in either the same or in different formulation(s).

In any of the forgoing aspects of the invention, the peripheral opioid antagonist can be selected from the group consisting of a piperidine-N-alkylcarboxylate, an opium alkaloid derivative, a quaternary benzomorphan compound, and a quaternary derivative of noroxymorphone. The preferred opioid antagonists are the quaternary derivatives of noroxymorphone, with the most preferred being methylnaltrexone.

In any of the forgoing aspects of the invention, the patients amenable to treatment are patients having the symptom of constipation and/or gastrointestinal immotility and who have failed to obtain relief of their symptoms using a laxative or a stool softener, either alone or in combination.

In any of the forgoing aspects of the invention, the peripheral opioid antagonist is a quaternary derivative of noroxymorphone and the patient is administered the peripheral opioid antagonist parentally in an amount ranging from 0.001 to 1.0 mg/kg.

In any of the forgoing aspects of the invention, the peripheral opioid antagonist is methylnaltrexone and the patient is administered the methylnaltrexone parentally in an amount ranging from 0.1 to 0.45 mg/kg. The amount can be from 0.1 to 0.3 mg/kg.

In any of the forgoing aspects of the invention, the peripheral opioid antagonist can be administered by any acceptable mode, parenterally, or not. Particular modes include, but are not limited to, intravenous, subcutaneous, needleless injection, rectal, or oral. In the case of oral administration, the peripheral opioid antagonist can be a quaternary derivative of noroxymorphone and the peripheral opioid antagonist can be administered in an amount ranging from 10 to 500 mg/kg, from 50 to 250 mg, or from 75 to 225 mg. If the administration route is oral, then the peripheral opioid antagonist can be administered in an enteric coated formulation.

According to another aspect of the invention, a formulation is provided which is a peripheral opioid antagonist and a laxative, a peripheral opioid antagonist and a stool softener, or a peripheral opioid antagonist and both a laxative and a stool softener. In one embodiment, the opioid antagonist and the laxative and/or stool softener are formulated as a suppository. In one embodiment, the peripheral opioid antagonist forms a core of a suppository. In one embodiment, the peripheral opioid antagonist is distributed throughout a suppository.

In one embodiment, the peripheral opioid antagonist is coated with a pharmaceutically acceptable carrier. In one embodiment, the peripheral opioid antagonist comprises particles. In one embodiment, peripheral opioid antagonist comprises particles and the particles are coated with a pharmaceutically acceptable carrier.

In one embodiment, the formulation is an oral formulation. In one embodiment, the formulation is an oral formulation and the peripheral opioid antagonist forms a core of the oral preparation. In one embodiment, the formulation is an oral formulation and the peripheral opioid antagonist is distributed throughout the oral formulation.

In one embodiment, at least a portion of the peripheral opioid antagonist is coated with a pharmaceutically acceptable carrier. In one embodiment, the pharmaceutically acceptable carrier is an enteric coating. In other embodiments, the peripheral opioid antagonist is not enterically coated.

In one embodiment, at least a portion of the laxative and/or stool softener is coated with a pharmaceutically acceptable carrier. In one embodiment, the pharmaceutically acceptable carrier is an enteric coating. In other embodiments, the laxative and/or stool softener is/are not enterically coated.

Any of the forgoing formulations can be constructed and arranged to release the peripheral opioid antagonist selectively anywhere along the gastrointestinal tract, such as in all of stomach, small intestine, and colon. Likewise, any can be constructed and arranged to release the peripheral opioid antagonist only in the small intestine and colon, only in the small intestine or only in the colon. Likewise, any can be constructed and arranged to release immediately substantially all of the peripheral opioid antagonist in the stomach.

In one embodiment, one or both of the peripheral opioid antagonist and the laxative and/or stool softener can be in a sustained release material. In other embodiments, one or both of the peripheral opioid antagonist and the laxative and/or stool softener is/are not in a sustained release material.

In any of the forgoing aspects of the invention, the peripheral opioid antagonist can be selected from the group consisting of a piperidine-N-alkylcarboxylate, an opium alkaloid derivative, a quaternary benzomorphan compound, and a quaternary derivative of noroxymorphone. The preferred opioid antagonists are the quaternary derivatives of noroxymorphone, with the most preferred being methylnaltrexone.

In certain of the forgoing aspects of the invention, the peripheral opioid antagonist is a quaternary derivative of noroxymorphone and the formulation contains the peripheral opioid antagonist in an amount ranging from 0.001 to 1.0 mg/kg, from 0.1 to 0.45 mg/kg, or from 0.1 to 0.3 mg/kg. In other of the forgoing aspects of the invention, the peripheral opioid antagonist is a quaternary derivative of noroxymorphone and the peripheral opioid antagonist and the formulation contains an amount of the antagonist ranging from 10 to 500 mg/kg, from 50 to 250 mg, or from 75 to 225 mg.

Any of the formulations can contain, optionally, an opioid.

According to another aspect of the invention, a kit is provided. The kit is a package containing a preparation of a peripheral opioid antagonist ad instructions for administering to a subject the antagonist and a laxative and/or stool softener. The kit can also include a preparation of a laxative and/or a stool softener. The peripheral opioid antagonist and the laxative and/or stool softener may be in the same or different formulations. The kit may include any of the formulations described above or throughout the specification. The kit also may include an administration device for administering one or more of the preparations. The administration device can be any means useful in administering one of the preparations in the kit, such as a syringe, an enema set, an infusion set, an inhaler, a spray device, a tube, etc.

According to another aspect of the invention, a method of manufacture is provided. The method involves combining a peripheral opioid antagonist with a laxative and/or stool softener to provide a formulation according to the invention. The method can further comprise combining a pharmaceutically acceptable carrier and/or an opioid with the foregoing formulation. The antagonist, laxative, stool softener, opioid and carrier may be any of those described herein.

These and other aspects of the invention will be apparent from the detailed description below.

### BRIEF DESCRIPTION OF THE DRAWING

FIG. 1 illustrates a kit according to the invention.

### DETAILED DESCRIPTION OF THE INVENTION

The subjects treatable according to the invention are human subjects suffering from constipation, gastrointestinal immotility, or other conditions calling for laxative or stool softener therapy. Constipation may be either chronic constipation or occasional constipation Constipation is characterized by less than one bowel movement in the previous three days or less than three bowel movements in the previous week (O'Keefe et al., J Gerontol., 50:184-189 (1995); Parup et al., Scand. J. Gastroenterol, 33:28-31 (1998)). Gastrointestinal immotility can include constipation, and also includes delayed oral cecal transit time, irregular Taxation, and other related gastrointestinal motility disfunction including impaction. Impaction is a condition where a large mass of dry, hard stool develops in the rectum, often due to chronic constipation. This mass may be so hard that it cannot be excreted. The subjects affected by constipation or gastrointestinal immotility can be refractory to laxative therapy and/or stool softener therapy. The subjects are treated with a combination of a peripheral opioid antagonist and a laxative, a peripheral opioid antagonist and a stool softener, or a peripheral opioid antagonist, a laxative and a stool softener.

The subjects may be experiencing constipation or gastrointestinal immotility due to known or unknown causes. The subjects may be constipated due to the presence of unwanted levels of endogenous opioids or due to exogenous opioid treatment. The subjects may be post-surgical subjects, subjects receiving opioids for pain, advanced medical illness subjects, terminally ill subjects, cancer patients, and the like. The subjects may have ileus.

Many other conditions call for laxative or stool softener therapy. Non-saline laxatives are used for: relief during pregnancy; relief a few days following giving birth; following surgery when straining should be avoided; following a period of poor eating habits or a lack of physical exercise which resulted in poor bowel habits (bulk forming laxatives only); medical conditions that may be made worse by straining during defecation (e.g., heart disease including angina, history of myocardial infarction, hemorrhoids; and hernia (rupture-type), hypertension, or history of stroke. Saline laxatives are used for: clearing the GI tract in preparation for examination or surgery (e.g., preparations of sodium di- and monohydrogen phosphate, preparations containing magnesium citrate with or without other salts, preparations of sodium sulfate and other salts, preparations of sodium sulfate and polyethylene glycol 3350); elimination of food or drugs from the body in cases of poisoning or overdose; and supplying a fresh stool sample for diagnosis. Bulk-forming laxatives are used for: treatment of hypercholesterolemia and lowering of plasma lipid levels (example, preparations of isfagula hulk). Certain laxatives can be used to get rid of worms. Certain laxatives can be used for reducing the amount of ammonia in the blood in conditions of hyperammonia (Lactulose is used for this purpose). Peripheral opioid antagonists are well-known in the art. Peripheral opioid antagonists, as used herein, means those which do not effectively cross the blood-brain barrier into the central nervous system. The majority of currently known opioid antagonists act both centrally and peripherally, and have potential for centrally mediated, undesirable side-effects. Naloxone and naltrexone are examples. The present invention involves the art recognized group of compounds known as peripheral opioid antagonists.

In preferred form, the methods of the present invention involve administering to a patient a compound which is a peripheral mu opioid antagonist compound. The term peripheral designates that the compound acts primarily on physiological systems and components external to the central nervous system, i.e., the compound does not readily cross the blood-brain barrier. The peripheral mu opioid antagonist compounds employed in the methods of the present invention typically exhibit high levels of activity with respect to gastrointestinal tissue, while exhibiting reduced, and preferably substantially no, central nervous system (CNS) activity. The term “substantially no CNS activity”, as used herein, means that less than about 20% of the pharmacological activity of the peripheral mu opioid antagonist compounds employed in the present methods is exhibited in the CNS. In preferred embodiments, the peripheral mu opioid antagonist compounds employed in the present methods exhibit less than about 5% of their pharmacological activity in the CNS, with about 1% or less (i.e., no CNS activity) being still more preferred.

The peripheral opioid antagonist may be, for example, a piperidine-N-alkylcarboxylate such as described in U.S. Pat. Nos. 5,250,542; 5,434,171; 5,159,081; 5,270,328; and 6,469,030. It also may be an opium alkaloid derivative such as described in U.S. Pat. Nos. 4,730,048; 4,806,556; and 6,469,030. Other peripheral opioid antagonists include quaternary benzomorphan compounds such as described in U.S. Pat. Nos. 3,723,440 and 6,469,030. The preferred antagonists are quaternary derivatives of noroxymorphone such as methylnaltrexone, described in U.S. Pat. Nos. 4,176,186 and 5,972,954. Other examples of quaternary derivatives of noroxymorphone include methylnaloxone and methylnalorphine.

A particularly preferred quaternary derivative of noroxymorphone is methylnaltrexone and salts thereof, described first by Goldberg, et al. Methylnaltrexone is also described in U.S. Pat. Nos. 4,719,215; 4,861,781; 5,102,887; 6,274,591; U.S. patent application Nos. 2002/0028825 and 2003/0022909; and PCT publication Nos. WO 99/22737 and WO 98/25613; each hereby incorporated by reference. As used herein, “methylnaltrexone” includes N-methylnaltrexone and salts thereof. Salts include, but are not limited to, bromide salts, chloride salts, iodide salts, carbonate salts, and sulfate salts.

Methylnaltrexone is provided as a white crystalline powder freely soluble in water. Its melting point is 254-256 ° C. Methylnaltrexone is available in a powder form from Mallinckrodt Pharmaceuticals, St. Louis, Mo. The compound as provided is 99.4% pure by reverse phase HPLC, and contains less than 0.011% unquaternized naltrexone by the same method. Methylnaltrexone is also identified as N-methyl-naltrexone bromide, naltrexone methobromide, N-methylnaltrexone, MNTX, SC-37359, MRZ-2663-BR, and N-cyclopropylmethylnoroxy-morphine-methobromide.

The peripheral opioid antagonists are administered with laxatives. Laxatives are well known to those of ordinary skill in the art and include a variety of different agents. Categories of laxatives include, but are not limited to, cathartic laxatives, bulk forming laxatives, diphenylmethane laxatives, hyperosmotic laxatives, mineral oils, and ‘saline’ laxatives. Specific examples are as follows.

Cathartic laxatives: aloe and related preparations and extracts from species of the genus Aloe; cascara sagrada and related preparations and extracts of the species Rhamnus purshiana including casanthranol; frangula and related preparations and extracts of the species Rhamnus frangula; senna and related preparations and extracts of species of the genus Cassia; sennosides A and B and combinations thereof; concentrated solutions of the above; combinations of the above.

Bulk forming laxatives: methylcellulose; carboxymethylcellulose sodium; karaya and related preparations from species of the genuses Sterculia or Cochlospermum, malt soup extract; psyllium and related preparations and extracts of species of the genus Plantago including psyllium hydrophilic mucilloid; combinations of the above.

Diphenylmethane laxatives: bisacodyl; bisacodyl tannex; phenolphthalein; diphenylmethane derivatives; combinations of the above including, optionally, magnesium salts such as magnesium citrate or sodium phosphate buffers.

Hyperosmotic laxatives: glycerin (glycerol); sorbitol (d-glucitol).

Mineral oils: heavy liquid petrolatum; heavy mineral oil; liquid paraffin; white mineral oil.

Saline laxatives: magnesium citrate; magnesium hydroxide; magnesium sulfate; magnesium oxide; sodium phosphate; mono- and di-basic sodium phosphate; potassium bitartrate and sodium bicarbonate.

The peripheral opioid antagonists are administered with stool softeners. Stool softeners are well known to those of ordinary skill in the art and include a variety of different agents. Stool softeners include, but are not limited to, docusate calcium (dioctyl calcium sulfosuccinate); docusate potassium (dioctyl potassium sulfosuccinate) and docusate sodium.

Other laxatives or stool softeners include castor oil, dehydrocholic acid, lactulose, polyethylene glycols, polyethylene glycol 3350, guiafensin, poloxamer 188 (a copolymer consisting of poly(ethylene oxide)-poly(propylene oxide)-poly(ethylene oxide) in a weight ratio of 4:2:4), herbal teas, 1,8-dihydroxyanthraquinone, polycarbophil, soy milk, caffeine, and bentonite clay.

The pharmaceutical preparations of the invention, when used in alone or in cocktails, are administered in therapeutically effective amounts. A therapeutically effective amount will be determined by the parameters discussed below; but, in any event, is that amount which establishes a level of the drug(s) effective for treating a subject, such as a human subject, having one of the conditions described herein. An effective amount means that amount alone or with multiple doses, necessary to delay the onset of, lessen the severity of, or inhibit completely, lessen the progression of, or halt altogether the onset or progression of the condition being treated or a symptom associated therewith. In the case of constipation, an effective amount, for example, is that amount which relieves a symptom of constipation, which induces a bowel movement such as by inducing laxation, which increases the frequency of bowel movements, or which otherwise decreases oral-cecal transit time. When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation.

Generally, oral doses of the quaternary derivatives of noroxymorphone will be from about 0.25 to about 5.0 mg/kg body weight per day. It is expected that oral doses in the range from 0.5 to 5.0 mg/kg body weight will yield the desired results. Generally, parenteral administration, including intravenous and subcutaneous administration, will be from about 0.001 to 1.0 mg/kg body weight. It is expected that doses ranging from 0.001 to 0.45 mg/kg body weight will yield the desired results, and doses of 0.1 to 0.3 are preferred. It is expected that infusion doses in the range from 0.001 to 1 mg/kg body weight will yield the desired results. Dosage may be adjusted appropriately to achieve desired drug levels, local or systemic, depending on the mode of administration. For example, it is expected that the dosage for oral administration of the opioid antagonists in an enterically-coated formulation would be lower than in an immediate release oral formulation. In the event that the response in a patient is insufficient at such doses, even higher doses (or effectively higher dosage by a different, more localized delivery route) may be employed to the extent that the patient tolerance permits. Multiple doses per day are contemplated to achieve appropriate systemic levels of compounds. Appropriate system levels can be determined by, for example, measurement of the patient's peak or sustained plasma level of the drug. “Dose” and “dosage” are used interchangeably herein.

Doses for laxatives, stool softeners and opioids are well characterized and known to those of ordinary skill in the art.

A variety of administration routes are available. The particular mode selected will depend, of course, upon the particular combination of drugs selected, the severity of the constipation or gastrointestinal immotility being treated, or prevented, the condition of the patient, and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, intravenous infusion, pulmonary, intramuscular, intracavity, aerosol, aural (e.g., via eardrops), intranasal, inhalation, needleless injection, subcutaneous or intradermal (e.g., transdermal) delivery. Direct injection could also be preferred for local delivery. For continuous infusion, a patient-controlled analgesia (PCA) device may be employed. Oral, rectal or subcutaneous administration may be important for prophylactic or long-term treatment. Preferred rectal modes of delivery include administration as a suppository or enema wash.

The pharmaceutical preparations may conveniently be presented in unit dosage form and may be prepared by any of the methods well known in the art of pharmacy. All methods include the step of bringing the compounds of the invention into association with a carrier which constitutes one or more accessory ingredients. In general, the compositions are prepared by uniformly and intimately bringing the compounds of the invention into association with a liquid carrier, a finely divided solid carrier, or both, and then, if necessary, shaping the product.

When administered, the pharmaceutical preparations of the invention are applied in pharmaceutically acceptable compositions. Such preparations may routinely contain salts, buffering agents, preservatives, compatible carriers, lubricants and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluenesulfonic, tartaric, citric, methanesulfonic, formic, succinic, naphthalene-2-sulfonic, pamoic, 3-hydroxy-2-naphthalenecarboxylic, and benzene sulfonic.

The pharmaceutical preparations of the present invention may include or be diluted into a pharmaceutically-acceptable carrier. The term “pharmaceutically-acceptable carrier” as used herein means one or more compatible solid or liquid fillers, diluents or encapsulating substances which are suitable for administration to a human or other mammal such as a dog, cat, horse, cow, sheep, or goat. The term “carrier” denotes an organic or inorganic ingredient, natural or synthetic, with which the active ingredient is combined to facilitate the application. The carriers are capable of being commingled with the preparations of the present invention, and with each other, in a manner such that there is no interaction which would substantially impair the desired pharmaceutical efficacy or stability. Carrier formulations suitable for oral administration, for suppositories, and for parenteral administration, etc., can be found in Remington's Pharmaceutical Sciences, Mack Publishing Company, Easton, Pa.

Aqueous formulations may include a chelating agent, a buffering agent, an anti-oxidant and, optionally, an isotonicity agent, preferably pH adjusted to between 3.0 and 3.5. Preferred such formulations that are stable to autoclaving and long term storage are described in co-pending application Ser. No. 60/461,611, filed on the same date hereof, entitled “Pharmaceutical Formulation,” the disclosure of which is incorporated herein by reference.

Chelating agents include: ethylenediaminetetraacetic acid (EDTA) and derivatives thereof, citric acid and derivatives thereof, niacinamide and derivatives thereof, sodium desoxycholate and derivatives thereof.

Buffering agents include those selected from the group consisting of citric acid, sodium citrate, sodium acetate, acetic acid, sodium phosphate and phosphoric acid, sodium ascorbate, tartaric acid, maleic acid, glycine, sodium lactate, lactic acid, ascorbic acid, imidazole, sodium bicarbonate and carbonic acid, sodium succinate and succinic acid, histidine, and sodium benzoate and benzoic acid, or any combination thereof.

Antioxidants include those selected from the group consisting of an ascorbic acid derivative, butylated hydroxy anisole, butylated hydroxy toluene, alkyl gallate, sodium meta-bisulfite, sodium bisulfite, sodium dithionite, sodium thioglycollate acid, sodium formaldehyde sulfoxylate, tocopheral and derivatives thereof, monothioglycerol, and sodium sulfite. The preferred antioxidant is monothioglycerol.

Isotonicity agents include those selected from the group consisting of sodium chloride, mannitol, lactose, dextrose, glycerol, and sorbitol.

Preservatives that can be used with the present compositions include benzyl alcohol, parabens, thimerosal, chlorobutanol and preferably benzalkonium chloride. Typically, the preservative will be present in a composition in a concentration of up to about 2% by weight. The exact concentration of the preservative, however, will vary depending upon the intended use and can be easily ascertained by one skilled in the art.

Patients particularly amenable to treatment are patients having the symptoms of constipation and/or gastrointestinal immotility and who have failed to obtain relief or ceased to obtain relief or a consistent degree of relief of their symptoms using a laxative or a stool softener, either alone or in combination, or who are otherwise resistant to laxatives and/or stool softeners. Such patients are said to be refractory to the conventional laxatives and/or stool softeners. The constipation and/or gastrointestinal immotility may be induced or a consequence of one or more diverse conditions including, but not limited to, a disease condition, a physical condition, a drug-induced condition, a physiological imbalance, stress, anxiety, and the like. The conditions inducing constipation and/or gastrointestinal immotility may be acute conditions or chronic conditions. In one embodiment, the constipation and/or gastrointestinal immotility results from opioid therapy provided for relief of pain. In one example of this embodiment, a human subject is experiencing the constipation and/or gastrointestinal immotility due to chronic opioid use.

Patients amenable to the therapy of the present invention include but are not limited to terminally ill patients, patients with advanced medical illness, cancer patients, AIDS patients, post-operative patients, patients with chronic pain, patients with neuropathies, patients with rheumatoid arthritis, patients with osteoarthritis, patients with chronic back pain, patients with spinal cord injury, patients with chronic abdominal pain, patients with chronic pancreatic pain, patients with pelvic/perineal pain, patients with fibromyalgia, patients with chronic fatigue syndrome, patients with irritable bowel syndrome, patients with migraine or tension headaches, patients on hemodialysis, and the like.

The subjects can be treated with a combination of the peripheral opioid antagonist and a laxative and/or a stool softener (and optionally, an opioid). In these circumstances the opioid antagonist and the other therapeutic agent(s) are administered close enough in time is such that the subject experiences the effects of the various agents as desired, which typically is at the same time. In some embodiments the opioid antagonist will be delivered first in time, in some embodiments second in time, and still in some embodiments at the same time. As discussed in greater detail below, the invention contemplates pharmaceutical preparations where the opioid antagonist is administered in a formulation including the opioid antagonist and one or both of a laxative and a stool softener (and, optionally, an opioid). These formulations may be parenteral or oral, such as the formulations described in U.S. Pat. Nos. 6,277,384; 6,261,599; 5,958,452; and PCT publication No. WO 98/25613, each hereby incorporated by reference. Included are solid, semisolid, liquid, controlled release and other such formulations.

A product containing an opioid antagonist and one or more other active agents can be configured as an oral dosage. The oral dosage may be a liquid, a semisolid or a solid. The oral dosage can include an opioid antagonist together with a laxative or a stool softener. An opioid may optionally be included in the oral dosage. The oral dosage may be configured to release the opioid antagonist before, after or simultaneously with the laxative or stool softener (and/or the opioid). The oral dosage may be configured to have the opioid antagonist and the other agents release completely in the stomach, release partially in the stomach and partially in the intestine, in the intestine, in the colon, partially in the stomach, or wholly in the colon. The oral dosage also may be configured whereby the release of the opioid antagonist is confined to the stomach or intestine while the release of the other active agent is not so confined or is confined differently from the opioid antagonist. For example, the opioid antagonist may be an enterically coated core or pellets contained within a pill or capsule that releases the laxative or stool softener first and releases the opioid antagonist only after the opioid antagonist passes through the stomach and into the intestine. The opioid antagonist also can be in a sustained release material, whereby the opioid antagonist is released throughout the gastrointestinal tract and the laxative or stool softener is released on the same or a different schedule. The same objective for opioid antagonist release can be achieved with immediate release of opioid antagonist combined with enteric coated opioid antagonist. In these instances, the laxative or stool softener could be released immediately in the stomach, throughout the gastrointestinal tract or only in the intestine.

The materials useful for achieving these different release profiles are well known to those of ordinary skill in the art. Immediate release is obtainable by conventional tablets with binders which dissolve in the stomach. Coatings which dissolve at the pH of the stomach or which dissolve at elevated temperatures will achieve the same purpose. Release only in the intestine is achieved using conventional enteric coatings such as pH sensitive coatings which dissolve in the pH environment of the intestine (but not the stomach) or coatings which dissolve over time. Release throughout the gastrointestinal tract is achieved by using sustained-release materials and/or combinations of the immediate release systems and sustained and/or delayed intentional release systems (e.g., pellets which dissolve at different pHs).

A product containing both an opioid antagonist and an irritable bowel syndrome (IBS) therapeutic agent also can be configured as a suppository. The opioid antagonist can be placed anywhere within or on the suppository to favorably affect the relative release of the opioid antagonist. The nature of the release can be zero order, first order, or sigmoidal, as desired.

In the event that it is desirable to release the opioid antagonist first, the opioid antagonist could be coated on the surface of the suppository in any pharmaceutically acceptable carrier suitable for such coatings and for permitting the release of the opioid antagonist, such as in a temperature sensitive pharmaceutically acceptable carrier used for suppositories routinely. Other coating which dissolve when placed in a body cavity are well known to those of ordinary skill in the art.

The opioid antagonist also may be mixed throughout the suppository, whereby it is released before, after or simultaneously with the laxative or stool softener. The opioid antagonist may be free, that is, solubilized within the material of the suppository. The opioid antagonist also may be in the form of vesicles, such as wax coated micropellets dispersed throughout the material of the suppository. The coated pellets can be fashioned to immediately release the opioid antagonist based on temperature, pH or the like. The pellets also can be configured so as to delay the release of the opioid antagonist, allowing the laxative or stool softener a period of time to act before the opioid antagonist exerts its effects. The opioid antagonist pellets also can be configured to release the opioid antagonist in virtually any sustained release pattern, including patterns exhibiting first order release kinetics or sigmoidal order release kinetics using materials of the prior art and well known to those of ordinary skill in the art.

The opioid antagonist also can be contained within a core within the suppository. The core may have any one or any combination of the properties described above in connection with the pellets. The opioid antagonist may be, for example, in a core coated with a material, dispersed throughout a material, coated onto a material or adsorbed into or throughout a material.

It should be understood that the pellets or core may be of virtually any type. They may be drug coated with a release material, drug interspersed throughout material, drug adsorbed into a material, and so on. The material may be erodible or nonerodible.

The suppository optionally can contain an opioid. The opioid can be in any of the forms described above in connection with the opioid antagonist but separate from the opioid antagonist. The opioid also may be mixed together with the opioid antagonist and provided in any of the forms described above in connection with opioid antagonist.

Oral and suppository formulations of laxatives and suppositories are well known and commercially available. The peripheral opioid antagonist can be added to such well known formulations. The peripheral opioid antagonist can be mixed together in solution or semi-solid solution in such formulations, can be provided in a suspension within such formulations or could be contained in particles within such formulations.

The therapeutic agent(s), including specifically but not limited to the peripheral opioid antagonist, may be provided in particles. Particles as used herein means nano or microparticles (or in some instances larger) which can consist in whole or in part of the peripheral opioid antagonists or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the antagonist in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as nonimmediate release formulations, with nonimmediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.”

Delivery systems specific for the gastrointestinal tract are roughly divided into three types: the first is a delayed release system designed to release a drug in response to with, for example, a change in pH; the second is a timed-release system designed to release a drug after a predetermined time; and the third is a microflora enzyme system making use of the abundant enterobacteria in the lower part of the gastrointestinal tract (e.g., in a colonic site-directed release formulation).

An example of a delayed release system is one that uses, for example, an acrylic or cellulosic coating material and dissolves on pH change. Because of ease of preparation, many reports on such “enteric coatings” have been made. In general, an enteric coating is one which passes through the stomach without releasing substantial amounts of drug in the stomach (i.e., less than 10% release, 5% release and even 1% release in the stomach) and sufficiently disintegrating in the intestine tract (by contact with approximately neutral or alkaline intestine juices) to allow the transport (active or passive) of the active agent through the walls of the intestinal tract.

Various in vitro tests for determining whether or not a coating is classified as an enteric coating have been published in the pharmacopoeia of various countries. A coating which remains intact for at least 2 hours, in contact with artificial gastric juices such as HCl of pH 1 at 36 to 38° C. and thereafter disintegrates within 30 minutes in artificial intestinal juices such as a KH₂PO₄ buffered solution of pH 6.8 is one example. One such well known system is EUDRAGIT material, commercially available and reported on by Boehringer, Manchester University, Saale Co., and the like. Enteric coatings are discussed further, below.

A timed release system is represented by Time Erosion System (TES) by Fujisawa Pharmaceutical Co., Ltd. and Pulsincap by R. P. Scherer. According to these systems, the site of drug release is decided by the time of transit of a preparation in the gastrointestinal tract. Since the transit of a preparation in the gastrointestinal tract is largely influenced by the gastric emptying time, some time release systems are also enterically coated.

Systems making use of the enterobacteria can be classified into those utilizing degradation of azoaromatic polymers by an azo reductase produced from enterobacteria as reported by the group of Ohio University (M. Saffran, et al., Science, Vol. 233: 1081 (1986)) and the group of Utah University (J. Kopecek, et al., Pharmaceutical Research, 9(12), 1540-1545 (1992)); and those utilizing degradation of polysaccharides by beta-galactosidase of enterobacteria as reported by the group of Hebrew University (unexamined published Japanese patent application No. 5-50863 based on a PCT application) and the group of Freiberg University (K. H. Bauer et al., Pharmaceutical Research, 10(10), S218 (1993)). In addition, the system using chitosan degradable by chitosanase by Teikoku Seiyaku K. K. (unexamined published Japanese patent application No. 4-217924 and unexamined published Japanese patent application No. 4-225922) is also included.

The enteric coating is typically although not necessarily a polymeric material. Preferred enteric coating materials comprise bioerodible, gradually hydrolyzable and/or gradually water-soluble polymers. The “coating weight,” or relative amount of coating material per capsule, generally dictates the time interval between ingestion and drug release. Any coating should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention The selection of the specific enteric coating material will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; ease of application as a coating (substrate friendly); and economical practicality.

Suitable enteric coating materials include, but are not limited to: cellulosic polymers such as cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl methacrylate (e.g., those copolymers sold under the tradename EUDRAGIT); vinyl polymers and copolymers such as polyvinyl acetate, polyvinylacetate phthalate, vinylacetate crotonic acid copolymer, and ethylene-vinyl acetate copolymers; and shellac (purified lac). Combinations of different coating materials may also be used. Well known enteric coating material for use herein are those acrylic acid polymers and copolymers available under the tradename EUDRAGIT from Rohm Pharma (Germany). The EUDRAGIT series E, L, S, RL, RS and NE copolymers are available as solubilized in organic solvent, as an aqueous dispersion, or as a dry powder. The EUDRAGIT series RL, NE, and RS copolymers are insoluble in the gastrointestinal tract but are permeable and are used primarily for extended release. The EUDRAGIT series E copolymers dissolve in the stomach. The EUDRAGIT series L, L-30D and S copolymers are insoluble in stomach and dissolve in the intestine, and are thus most preferred herein.

A particular methacrylic copolymer is EUDRAGIT L, particularly L-30D and EUDRAGIT L100-55. In EUDRAGIT L-30D, the ratio of free carboxyl groups to ester groups is approximately 1:1. Further, the copolymer is known to be insoluble in gastrointestinal fluids having pH below 5.5, generally 1.5-5.5, i.e., the pH generally present in the fluid of the upper gastrointestinal tract, but readily soluble or partially soluble at pH above 5.5, i.e., the pH generally present in the fluid of lower gastrointestinal tract. Another particular methacrylic acid polymer is EUDRAGIT S, which differs from EUDRAGIT L-30D in that the ratio of free carboxyl groups to ester groups is approximately 1:2. EUDRAGIT S is insoluble at pH below 5.5, but unlike EUDRAGIT L-30D, is poorly soluble in gastrointestinal fluids having a pH in the range of 5.5 to 7.0, such as in the small intestine. This copolymer is soluble at pH 7.0 and above, i.e., the pH generally found in the colon. EUDRAGIT S can be used alone as a coating to provide drug delivery in the large intestine. Alternatively, EUDRAGIT S, being poorly soluble in intestinal fluids below pH 7, can be used in combination with EUDRAGIT L-30D, soluble in intestinal fluids above pH 5.5, in order to provide a delayed release composition which can be formulated to deliver the active agent to various segments of the intestinal tract. The more EUDRAGIT L-30D used, the more proximal release and delivery begins, and the more EUDRAGIT S used, the more distal release and delivery begins. It will be appreciated by those skilled in the art that both EUDRAGIT L-30D and EUDRAGIT S can be replaced with other pharmaceutically acceptable polymers having similar pH solubility characteristics.

In certain embodiments of the invention, the preferred enteric coating is ACRYL-EZE™ (methacrylic acid co-polymer type C; Colorcon, West Point, Pa.).

The enteric coating provides for controlled release of the active agent, such that drug release can be accomplished at some generally predictable location. The enteric coating also prevents exposure of the therapeutic agent and carrier to the epithelial and mucosal tissue of the buccal cavity, pharynx, esophagus, and stomach, and to the enzymes associated with these tissues. The enteric coating therefore helps to protect the active agent, carrier and a patient's internal tissue from any adverse event prior to drug release at the desired site of delivery. Furthermore, the coated material of the present invention allow optimization of drug absorption, active agent~protection, and safety. Multiple enteric coatings targeted to release the active agent at various regions in the gastrointestinal tract would enable even more effective and sustained improved delivery throughout the gastrointestinal tract.

The coating can, and usually does, contain a plasticizer to prevent the formation of pores and cracks that would permit the penetration of the gastric fluids. Suitable plasticizers include, but are not limited to, triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating comprised of an anionic carboxylic acrylic polymer will usually contain approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g., hydroxypropylcellulose, acids and bases) to solubilize or disperse the coating material, and to improve coating performance and the coated product. The coating can be applied to particles of the therapeutic agent(s), tablets of the therapeutic agent(s), capsules containing the therapeutic agent(s) and the like, using conventional coating methods and equipment. For example, an enteric coating can be applied to a capsule using a coating pan, an airless spray technique, fluidized bed coating equipment, or the like. Detailed information concerning materials, equipment and processes for preparing coated dosage forms may be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6th Ed. (Media, Pa.: Williams & Wilkins, 1995). The coating thickness, as noted above, must be sufficient to ensure that the oral dosage form remains intact until the desired site of topical delivery in the lower intestinal tract is reached.

In another embodiment, drug dosage forms are provided that comprise an enterically coated, osmotically activated device housing a formulation of the invention. In this embodiment, the drug-containing formulation is encapsulated in a semipermeable membrane or barrier containing a small orifice. As known in the art with respect to so-called “osmotic pump” drug delivery devices, the semipermeable membrane allows passage of water in either direction, but not drug. Therefore, when the device is exposed to aqueous fluids, water will flow into the device due to the osmotic pressure differential between the interior and exterior of the device. As water flows into the device, the drug-containing formulation in the interior will be “pumped” out through the orifice. The rate of drug release will be equivalent to the inflow rate of water times the drug concentration. Suitable materials for the semipermeable membrane include, but are not limited to, polyvinyl alcohol, polyvinyl chloride, semipermeable polyethylene glycols, semipermeable polyurethanes, semipermeable polyamides, semipermeable sulfonated polystyrenes and polystyrene derivatives; semipermeable poly(sodium styrenesulfonate), semipermeable poly(vinylbenzyltrimethylammonium chloride), and cellulosic polymers such as cellulose acetate, cellulose diacetate, cellulose triacetate, cellulose propionate, cellulose acetate propionate, cellulose acetate butyrate, cellulose trivalerate, cellulose trilmate, cellulose tripalmitate, cellulose tripropionate, cellulose disuccinate, cellulose dipalmitate, cellulose dicarpylate, cellulose acetate succinate, cellulose propionate succinate, cellulose acetate octanoate, cellulose valerate palmitate, cellulose acetate heptanate, cellulose acetaldehyde dimethyl acetate, cellulose acetate ethylcarbamate, cellulose acetate methylcarbamate, cellulose dimethylaminoacetate and ethylcellulose.

In another embodiment, drug dosage forms are provided that comprise a sustained release coated device housing a formulation of the invention. In this embodiment, the drug-containing formulation is encapsulated in a sustained release membrane or film. The membrane may be semipermeable, as described above. Semipermeable membrane allow for the passage of water inside the coated device to dissolve the drug. The dissolved drug solution diffuses out through the semipermeable membrane. The rate of drug release depends upon the thickness of the coated film and the release of drug can begin in any part of the GI tract. Suitable membrane materials for such a membrane include ethyl cellulose.

In another embodiment, drug dosage forms are provided that comprise a sustained release device housing a formulation of the invention. In this embodiment, the drug-containing formulation is uniformly mixed with a sustained release polymer. These sustained release polymers are high molecular weight water-soluble polymers, which when in contact with water, swell and create channels for water to diffuse inside and dissolve the drug. As the polymers swell and dissolve in water, more of drug is exposed to water for dissolution. Such a system is generally referred to as sustained release matrix. Suitable materials for such a device include hydropropyl methylcellulose, hydroxypropyl cellulose, hydroxyethyl cellulose and methyl cellulose.

In another embodiment, drug dosage forms are provided that comprise an enteric coated device housing a sustained release formulation of the invention. In this embodiment, the drug containing product described above are coated with an enteric polymers. Such a device would not release any drug in the stomach and when the device reaches the intestine, the enteric polymer is first dissolved and only then would the drug release begin. The drug release would take place in a sustained release fashion.

Enterically coated, osmotically activated devices can be manufactured using conventional materials, methods and equipment. For example, osmotically activated devices may be made by first encapsulating, in a pharmaceutically acceptable soft capsule, a liquid or semi-solid formulation as described previously. This interior capsule is then coated with a semipermeable membrane composition (comprising, for example, cellulose acetate and polyethylene glycol 4000 in a suitable solvent such as a methylene chloride-methanol admixture), for example using an air suspension machine, until a sufficiently thick laminate is formed, e.g., around 0.05 mm. The semipermeable laminated capsule is then dried using conventional techniques. Then, an orifice having a desired diameter (e.g., about 0.99 mm) is provided through the semipermeable laminated capsule wall, using, for example, mechanical drilling, laser drilling, mechanical rupturing, or erosion of an erodible element such as a gelatin plug. The osmotically activated device may then be enterically coated as previously described. For osmotically activated devices containing a solid carrier rather than a liquid or semi-solid carrier, the interior capsule is optional; that is, the semipermeable membrane may be formed directly around the carrier-drug composition. However, preferred carriers for use in the drug-containing formulation of the osmotically activated device are solutions, suspensions, liquids, immiscible liquids, emulsions, sols, colloids, and oils. Particularly preferred carriers include, but are not limited to, those used for enterically coated capsules containing liquid or semisolid drug formulations.

Cellulose coatings include those of cellulose acetate phthalate and trimellitate; methacrylic acid copolymers, e.g. copolymers derived from methylacrylic acid and esters thereof, containing at least 40% methylacrylic acid; and especially hydroxypropyl methylcellulose phthalate. Methylacrylates include those of molecular weight above 100,000 daltons based on, e.g. methylacrylate and methyl or ethyl methylacrylate in a ratio of about 1:1. Typical products include EUDRAGIT L, e.g. L 100-55, marketed by Rohm GmbH, Darmstadt, Germany. Typical cellulose acetate phthalates have an acetyl content of 17-26% and a phthalate content of from 30-40% with a viscosity of ca. 45-90 cP. Typical cellulose acetate trimellitates have an acetyl content of 17-26%, a trimellityl content from 25-35% with a viscosity of ca. 15-20 cS. An example of a cellulose acetate trimellitate is the marketed product CAT (Eastman Kodak Company, USA). Hydroxypropyl methylcellulose phthalates typically have a molecular weight of from 20,000 to 130,000 daltons, a hydroxypropyl content of from 5 to 10%, a methoxy content of from 18 to 24% and a phthalyl content from 21 to 35%. An example of a cellulose acetate phthalate is the marketed product CAP (Eastman Kodak, Rochester N.Y., USA). Examples of hydroxypropyl methylcellulose phthalates are the marketed products having a hydroxypropyl content of from 6-10%, a methoxy content of from 20-24%, a phthalyl content of from 21-27%, a molecular weight of about 84,000 daltons, known under the trade mark HP50 and available from Shin-Etsu Chemical Co. Ltd., Tokyo, Japan, and having a hydroxypropyl content, a methoxyl content, and a phthalyl content of 5-9%, 18-22% and 27-35%, respectively, and a molecular weight of 78,000 daltons, known under the trademark HP55 and available from the same supplier.

The therapeutic agents may be provided in capsules, coated or not. The capsule material may be either hard or soft, and as will be appreciated by those skilled in the art, typically comprises a tasteless, easily administered and water soluble compound such as gelatin, starch or a cellulosic material. The capsules are preferably sealed, such as with gelatin bands or the like. See, for example, Remington: The Science and Practice of Pharmacy, Nineteenth Edition (Easton, Pa.: Mack Publishing Co., 1995), which describes materials and methods for preparing encapsulated pharmaceuticals.

The therapeutic agents may be provided in suppositories. Suppositories are solid dosage forms of medicine intended for administration via the rectum. Suppositories are compounded so as to melt, soften, or dissolve in the body cavity (around 98.6° F.) thereby releasing the medication contained therein. Suppository bases should be stable, nonirritating, chemically inert, and physiologically inert. Many commercially available suppositories contain oily or fatty base materials, such as cocoa butter, coconut oil, palm kernel oil, and palm oil, which often melt or deform at room temperature necessitating cool storage or other storage limitations. U.S. Pat. No. 4,837,214 to Tanaka et al. describes a suppository base comprised of 80 to 99 percent by weight of a lauric-type fat having a hydroxyl value of 20 or smaller and containing glycerides of fatty acids having 8 to 18 carbon atoms combined with 1 to 20 percent by weight diglycerides of fatty acids (which erucic acid is an example of). The shelf life of these type of suppositories is limited due to degradation. Other suppository bases contain alcohols, surfactants, and the like which raise the melting temperature but also can lead to poor absorption of the medicine and side effects due to irritation of the local mucous membranes (see for example, U.S. Pat. No. 6,099,853 to Hartelendy et al., U.S. Pat. No. 4,999,342 to Ahmad et al., and U.S. Pat. No. 4,765,978 to Abidi et al.).

The base used in the pharmaceutical suppository composition of this invention include, in general, oils and fats comprising triglycerides as main components such as cacao butter, palm fat, palm kernel oil, coconut oil, fractionated coconut oil, lard and WITEPSOL®, waxes such as lanolin and reduced lanolin; hydrocarbons such as Vaseline, squalene, squalane and liquid paraffin; long to medium chain fatty acids such as caprylic acid, lauric acid, stearic acid and oleic acid; higher alcohols such as lauryl alcohol, cetanol and stearyl alcohol; fatty acid esters such as butyl stearate and dilauryl malonate; medium to long chain carboxylic acid esters of glycerin such as triolein and tristearin; glycerin-substituted carboxylic acid esters such as glycerin acetoacetate; and polyethylene glycols and its derivatives such as macrogols and cetomacrogol. They may be used either singly or in combination of two or more. If desired, the composition of this invention may further include a surface active agent, a coloring agent, etc., which are ordinarily used in suppositories.

The pharmaceutical composition of this invention may be prepared by uniformly mixing predetermined amounts of the active ingredient, the absorption aid and optionally the base, etc. in a stirrer or a grinding mill, if required at an elevated temperature. The resulting composition, may be formed into a suppository in unit dosage form by, for example, casting the mixture in a mold, or by forming it into a gelatin capsule using a capsule filling machine.

The compositions according to the present invention also can be administered as a nasal spray, nasal drop, suspension, gel, ointment, cream or powder. The administration of a composition can also include using a nasal tampon or a nasal sponge containing a composition of the present invention.

The nasal delivery systems that can be used with the present invention can take various forms including aqueous preparations, non-aqueous preparations and combinations thereof. Aqueous preparations include, for example, aqueous gels, aqueous suspensions, aqueous liposomal dispersions, aqueous emulsions, aqueous microemulsions and combinations thereof. Non-aqueous preparations include, for example, non-aqueous gels, non-aqueous suspensions, non-aqueous liposomal dispersions, non-aqueous emulsions, non-aqueous microemulsions and combinations thereof. The various forms of the nasal delivery systems can include a buffer to maintain pH, a pharmaceutically acceptable thickening agent and a humectant. The pH of the buffer can be selected to optimize the absorption of the therapeutic agent(s) across the nasal mucosa.

With respect to the non-aqueous nasal formulations, suitable forms of buffering agents can be selected such that when the formulation is delivered into the nasal cavity of a mammal, selected pH ranges are achieved therein upon contact with, e.g., a nasal mucosa. In the present invention, the pH of the compositions should be maintained from about 2.0 to about 6.0. It is desirable that the pH of the compositions is one which does not cause significant irritation to the nasal mucosa of a recipient upon administration.

The viscosity of the compositions of the present invention can be maintained at a desired level using a pharmaceutically acceptable thickening agent. Thickening agents that can be used in accordance with the present invention include methyl cellulose, xanthan gum, carboxymethyl cellulose, hydroxypropyl cellulose, carbomer, polyvinyl alcohol, alginates, acacia, chitosans and combinations thereof. The concentration of the thickening agent will depend upon the agent selected and the viscosity desired. Such agents can also be used in a powder formulation discussed above.

The compositions of the present invention can also include a humectant to reduce or prevent drying of the mucus membrane and to prevent irritation thereof. Suitable humectants that can be used in the present invention include sorbitol, mineral oil, vegetable oil and glycerol; soothing agents; membrane conditioners; sweeteners; and combinations thereof. The concentration of the humectant in the present compositions will vary depending upon the agent selected.

One or more therapeutic agents may be incorporated into the nasal delivery system or any other delivery system described herein.

The formulations can be constructed and arranged to create steady state plasma levels. Steady state plasma concentrations can be measured using HPLC techniques, as are known to those of skill in the art. Steady state is achieved when the rate of drug delivery is equal to the rate of drug elimination from the circulation. In typical therapeutic settings, the quaternary derivatives of noroxymorphone will be administered to patients either on a periodic dosing regimen or with a constant infusion regimen. The concentration of drug in the plasma will tend to rise immediately after the onset of administration and will tend to fall over time as the drug is eliminated from the circulation by means of distribution into cells and tissues, by metabolism, or by excretion. Steady state will obtain when the mean drug concentration remains constant over time. In the case of intermittent dosing, the pattern of the drug concentration cycle is repeated identically in each interval between doses with the mean concentration remaining constant. In the case of constant infusion, the mean drug concentration will remain constant with very little oscillation. The achievement of steady state is determined by means of measuring the concentration of drug in plasma over at least one cycle of dosing such that one can verify that the cycle is being repeated identically from dose to dose. Typically, in an intermittent dosing regimen, maintenance of steady state can be verified by determining drug concentrations at the consecutive troughs of a cycle, just prior to administration of another dose. In a constant infusion regimen where oscillation in the concentration is low, steady state can be verified by any two consecutive measurements of drug concentration.

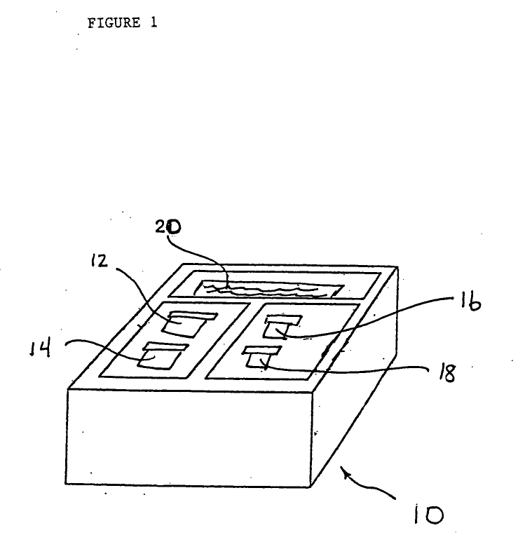

FIG. 1 shows a kit according to the invention. The kit 10 includes a laxative capsule 12 containing a laxative. The kit 10 also contains a methylnaltrexone capsule 14 containing methylnaltrexone pellets, some of which are enterically coated with pH sensitive material and some of which are constructed and arranged to release the methylnaltrexone immediately in the stomach. The kit also includes instructions for administering the capsules to a subject who is constipated or who has symptoms of constipation or gastrointestinal immotility.

In some aspects of the invention, the kit 10 can include a pharmaceutical preparation vial, a pharmaceutical preparation diluent vial, and a laxative and/or a stool softener. The vial containing the diluent for the pharmaceutical preparation is optional. The diluent vial contains a diluent such as physiological saline for diluting what could be a concentrated solution or lyophilized powder of methylnaltrexone. The instructions can include instructions for mixing a particular amount of the diluent with a particular amount of the concentrated pharmaceutical preparation, whereby a final formulation for injection or infusion is prepared. The instructions may include instructions for use in a PCA device. The instructions 20 can include instructions for treating a patient with an effective amount of methylnaltrexone. It also will be understood that the containers containing the preparations, whether the container is a bottle, a vial with a septum, an ampoule with a septum, an infusion bag, and the like, can contain indicia such as conventional markings which change color when the preparation has been autoclaved or otherwise sterilized.

All of the patents, applications and references referred to herein are incorporated by reference in their entirety.

The following Examples are illustrative of the invention and should not be construed as limitations of the invention.

### EXAMPLES

### 1. Example 1

### Manufacturing Details for Methylnaltrexone 75 mg Tablets (Non-Enteric)

| | mg per tablet |
|---|---|
| Ingredients used (Trade name) | |
| Methylnaltrexone | 75 |
| Microcrystalline cellulose | 13.30 |
| (Avicel PH 101) | |
| Polyvinylpyrrolidone | 3.5 |
| (Povidone K30) | |
| Croscarmellose sodium | 8 |
| (Ac-Di-Sol SD-711) | |
| Dibasic Calcium Phosphate | 199 |
| (Emcompress) | |
| Microcrystalline cellulose | 49.7 |
| (Avicel PH 200) | |
| Magnesium Stearate (Hyqual) | 1.7 |
| Opadry II Clear | 7.00 |
| Water | as needed |
| Equipment used | |
| Key KG-5 Granulator | to make granules . . . kind |
| | of dough maker |
| Glatt WSG-1, Uniglatt | to dry the granules |
| Quadro Comill | to break the granule particles |
| | to the desired size |
| Cross-Flow blender | to mix things together |
| Manesty beta-press | to compress powder into tablets |
| O'Hara Labcoat II-X | to coat the tablets with any film. |

Miscellaneous equipments such as balances, peristaltic pump,
propeller mixer and spatula etc.

Manufacturing steps:

2. Pass Methylnaltexone, Avicel 101 and Ac-Di-Sol (part of it) thru 20 mesh screen and add to the granulator.

3. Granulate the above mixture using a solution of Povidone in water.

4. After the granules are formed, transfer the material to Uniglatt and dry the mixture.

5. Repeat steps 1 to 3, two more times and combine the mixture. This was done due to equipment capacity being ⅓ of the total weight.

6. Pass the mixture in step #4 thru Comill.

7. Screen Avicel 200, Emcompress and the remaining Ac-Di-Sol thru 20 mesh screen and add it to the blender.

8. Add material from step #5 to material in step #6 and mix for 10 minutes.

9. Add Magnesium stearate to the blender and mix for 3 minutes.

10. Transfer the material to Manesty Beta-press and compress the tablets.

11. Coat the tablets with a solution of Opadry II Clear in water using a O'Hara Labcoat.

### 1. Example 2

### Manufacturing Details for Enteric Coating (Both 75 and 225 mg)

After step #9 from the previous example:

1. Coat the tablets with a suspension of Eudragit L in water.

2. Coat the material in step # 11 with Opadry white.

The polymer we will be using for the enteric part will be one of the following:

| | |
|---|---|
| Eudragit L | From Degussa or Rohm Pharma |
| Eudragit L 50D | From Degussa or Rohm Pharma |
| Acryl-eze (methacrylic acid | From Colorcon |
| co-polymer type C) | |
| Sureteric (polyvinyl acetate phthalate) | From Colorcon |

### Example 3

### Manufacturing Details for Oral Enterically Coated Sustained Release Tablets

Ingredients used:

| | |
|---|---|
| Methylnaltrexone | 250 g |
| Docusate sodium | 100 g |
| Lactose | 20 g |
| Hydroxypropyl methylcellulose (1000 cps) | 120 g |
| Polyvinylpyrrolidone | 10 g |
| Dibasic calcium phosphate | 50 g |
| Magnesium stearate | 3 g |
| Cellulose acetate phthalate | 50 g |
| Water | as needed |

Manufacturing steps:

1. Mix 250 g of methylnaltrexone with the 100 g of docusate sodium in a high shear blender.

2. Add 20 g of lactose and 120 g of hydroxypropyl methylcellulose to the blender and mix thoroughly.

3. Granulate the above mixture using a solution of polyvinylpyrrolidone in water (10 g in 100 ml).

4. After the granules are formed, transfer the material to a fluidized bed drier and dry the mixture.

5. Pass the mixture from step 4 through a mill to reduce the particle size of the granules to make it more uniform.

6. Add the material from step 5 to a tumble blender and add 50 g of dibasic calcium phosphate and mix thoroughly for 10 minutes.

7. Add 3 g of magnesium stearate to the blender and mix for 3 to 5 minutes.

8. Transfer the material to a tablet press and compress into tablets with a target weight of 553 mg per tablet.

9. Coat the tablets from step 8, in a perforated pan, with cellulose acetate phthalate to a tablet weight of 603 mg.

### Example 4

### Manufacturing Details for a Suppository:

Ingredients used:

| | |
|---|---|
| Methylnaltrexone | 250 g |
| Glycerin | 500 g |
| Polyethylene glycol 1000 | 100 g |
| Polyethylene glycol 4000 | 800 g |

Manufacturing steps:

1. In a jacketed pot, add 250 g of methylnaltrexone and 500 g of glycerin and start mixing.

2. Add 100 g of polyethylene glycol 1000 and 800 g of polyethylne glycol 4000 to the materials in step 1 and continue mixing.

3. The material from step 2 is heated via the jacket to render a flowable and pourable mixture.

4. The mixture is poured into containers for manufacturing suppositories and allowed to cool to room temperature.

5. Solidified suppositories are then harvested from the containers. Each suppository would weigh 1650 mg.

We claim:

1. A method for treating constipation comprising administering to a patient in need of such treatment a laxative and a peripheral opioid antagonist in amounts effective to treat the constipation.

2. The method of claim 1 wherein the patient is refractory to laxative therapy.

3. The method of claim 1 further comprising administering an opioid to the patient.

4. The method of claim 1 wherein the patient is receiving opioids chronically.

5. The method of claim 3 wherein the opioid is morphine.

6. The method of claim 1 wherein the peripheral opioid antagonist and laxative are administered in one formulation.

7. A method for treating constipation comprising administering to a patient in need of such treatment a stool softener and a peripheral opioid antagonist in amounts effective to treat the constipation.

8. The method of claim 7 wherein the patient is refractory to stool softener therapy.

9. The method of claim 7 further comprising administering an opioid to the patient.

10. The method of claim 9 wherein the opioid is administered chronically.

11. The method of claim 9 wherein the opioid is morphine.

12. The method of claim 7 wherein the peripheral opioid antagonist and stool softener are administered in one formulation.

13. A method for treating a condition calling for treatment with a laxative comprising administering to a patient in need of such treatment a laxative and a peripheral opioid antagonist in amounts effective to treat the condition.

14. The method of claim 13 wherein the patient is refractory to laxative therapy.

15. The method of claim 13 further comprising administering an opioid to the patient.

16. The method of claim 15 wherein the opioid is administered chronically.

17. The method of claim 15 wherein the opioid is morphine.

18. The method of claim 13 wherein the peripheral opioid antagonist and laxative are administered in one formulation.

19. A method for treating a condition for treatment with a stool softener comprising administering to a patient in need of such treatment a stool softener and a peripheral opioid antagonist in amounts effective to treat the condition.

20. The method of claim 19 wherein the patient is refractory to stool softener therapy.

21. The method of claim 19 further comprising administering an opioid to the patient.

22. The method of claim 21 wherein the opioid is administered chronically.

23. The method of claim 21 wherein the opioid is morphine.

24. The method of claim 19 wherein the stool softener peripheral opioid antagonist and stool softener are administered in one formulation.

25. The method of claim 1 wherein the peripheral opioid antagonist is a quaternary derivative of noroxymorphone.

26. The method of claim 25 wherein the peripheral opioid antagonist is methylnaltrexone.

27. The method of claim 1 wherein the patient is a terminally ill patient.

28. The method of claim 27 wherein the peripheral opioid antagonist is methylnaltrexone.

29. The method of claim 1 wherein the patient has an advanced medical illness.

30. The method of claim 29 wherein the peripheral opioid antagonist is methylnaltrexone.

31. The method of claim 1 wherein the patient is a cancer patient.

32. The method of claim 31 wherein the peripheral opioid antagonist is methylnaltrexone.

33. The method of claim 1 wherein the patient is a post-operative patient.

34. The method of claim 33 wherein the peripheral opioid antagonist is methylnaltrexone.

35. The method of claim 1 wherein the patient has chronic pain.

36. The method of claim 35 wherein the peripheral opioid antagonist is methylnaltrexone.

37. The method of claim 1 wherein the peripheral opioid antagonist is a quaternary derivative of noroxymorphone and the patient is administered the peripheral opioid antagonist parenterally in an amount ranging from 0.001 to 1.0 mg/kg.

38. The method of claim 37 wherein the peripheral opioid antagonist is methylnaltrexone and wherein the patient is administered the methylnaltrexone parenterally in an amount ranging from 0.1 to 0.45 mg/kg.

39. The method of claim 38 wherein the amount of methylnaltrexone ranges from 0.1 to 0.3 mg/kg.

40. The method of claim 38 wherein the peripheral opioid antagonist is administered parenterally.

41. The method of claim 40 wherein the peripheral opioid antagonist is administered via a route selected from the group consisting of intravenously, subcutaneously, and via a needleless injection.

42. The method of claim 1 wherein the patient is administered the peripheral opioid antagonist orally or rectally.

43. The method of claim 42 wherein the peripheral opioid antagonist is a quaternary derivative of noroxymorphone and the peripheral opioid antagonist is administered in an amount ranging from 10 to 500 mg/kg.

44. The method of claim 43 wherein the peripheral opioid antagonist is administered in an enteric coated formulation.

45. The method of claim 43 wherein the peripheral opioid antagonist is methylnaltrexone and wherein the patient is administered the methylnaltrexone orally in an amount ranging from 50 to 250 mg.

46. The method of claim 45 wherein the amount of methylnaltrexone ranges from 75 to 225 mg.

47. The method of claim 1 wherein the patient is administered the peripheral opioid antagonist rectally.

48. A formulation comprising a peripheral opioid antagonist and a laxative.

49. The formulation of claim 48 wherein the opioid antagonist and the laxative are formulated as a suppository.

50. The formulation of claim 49, wherein the peripheral opioid antagonist forms a core of the suppository.

51. The formulation of claim 49, wherein the peripheral opioid antagonist is distributed throughout the suppository.

52. The formulation of claim 49, wherein the peripheral opioid antagonist is coated with a pharmaceutically acceptable carrier.

53. The formulation of claim 49, wherein the peripheral opioid antagonist comprises particles.

54. The formulation of claim 51, wherein the particles are coated with a pharmaceutically acceptable carrier.

55. The formulation of claim 48, wherein the formulation is an oral formulation.

56. The formulation of claim 55, wherein the peripheral opioid antagonist forms a core of the oral preparation.

57. The formulation of claim 55, wherein the peripheral opioid antagonist is distributed throughout the oral formulation.

58. The formulation of claim 55, wherein at least a portion of the peripheral opioid antagonist is coated with a pharmaceutically acceptable carrier.

59. The formulation of claim 58, wherein the pharmaceutically acceptable carrier is an enteric coating.

60. The formulation of claim 59, wherein the laxative is not enterically coated.

61. The formulation of claim 55, wherein at least a portion of the laxative is coated with a pharmaceutically acceptable carrier.

62. The formulation of claim 61, wherein the pharmaceutically acceptable carrier is an enteric coating.

63. The formulation of claim 62, wherein the peripheral opioid antagonist is not enterically coated.

64. The formulation of claim 55, wherein the formulation is constructed and arranged to release the peripheral opioid antagonist in the stomach, the small intestine, and the colon.

65. The formulation of claim 55, wherein the formulation is constructed and arranged to release the peripheral opioid antagonist only in the small intestine and colon.

66. The formulation of claim 55, wherein the formulation is constructed and arranged to release the peripheral opioid antagonist only in the small intestine.

67. The formulation of claim 55, wherein the formulation is constructed and arranged to release the peripheral opioid antagonist only in the colon.

68. The formulation of claim 55, wherein the formulation is constructed and arranged to release immediately substantially all of the peripheral opioid antagonist in the stomach.

69. The formulation of claim 48, wherein the peripheral opioid antagonist is in or coated with a sustained release material.

70. The formulation of claim 48, wherein the peripheral opioid antagonist is in an enteric coated sustained release material.

71. The formulation of claim 69, wherein the laxative is not in a sustained release material.

72. The formulation of claim 48, wherein the laxative is in or coated with a sustained release material.

73. The formulation of claim 72, wherein the sustained release material is a matrix or membrane.

74. The formulation of claim 48, wherein the laxative is an enteric coated sustained release material.

75. The formulation of claim 72, wherein the peripheral opioid antagonist is not in a sustained release material.

76. The formulation of claim 48, wherein the peripheral opioid antagonist is a quaternary derivative of noroxymorphone.

77. The formulation of claim 76 wherein the peripheral opioid antagonist is methylnaltrexone.

78. The formulation of claim 77, wherein the methylnaltrexone is present in a range from 50 to 250 mg.

79. The formulation of claim 77, wherein the formulation further comprises an opioid.

80. A formulation comprising a peripheral opioid antagonist and a stool softener.

81. The formulation of claim 80, wherein the opioid antagonist and the stool softener are formulated as a suppository.

82. The formulation of claim 80, wherein the peripheral opioid antagonist forms a core of the suppository.

83. The formulation of claim 80, wherein the peripheral opioid antagonist is distributed throughout the suppository.

84. The formulation of claim 80, wherein the peripheral opioid antagonist is coated with a pharmaceutically acceptable carrier.

85. The formulation of claim 80, wherein the peripheral opioid antagonist comprises particles.

86. The formulation of claim 85, wherein the particles are coated with a pharmaceutically acceptable carrier.

87. The formulation of claim 80, wherein the formulation is an oral formulation.

88. The formulation of claim 87, wherein the formulation is a liquid, semi-solid or solid.

89. The formulation of claim 87, wherein the peripheral opioid antagonist forms a core of the oral preparation.

90. The formulation of claim 87, wherein the peripheral opioid antagonist is distributed throughout the oral formulation.

91. The formulation of claim 87, wherein at least a portion of the peripheral opioid antagonist is coated with a pharmaceutically acceptable carrier.

92. The formulation of claim 91, wherein the pharmaceutically acceptable carrier is an enteric coating.

93. The formulation of claim 91, wherein the pharmaceutically acceptable carrier is a sustained release coating.

94. The formulation of claim 93, wherein the stool softener is not enterically coated.

95. The formulation of claim 87, wherein at least a portion of the stool softener is coated with a pharmaceutically acceptable carrier.

96. The formulation of claim 95, wherein the pharmaceutically acceptable carrier is an enteric coating.

97. The formulation of claim 95, wherein the pharmaceutically acceptable carrier is a sustained release coating.

98. The formulation of claim 96, wherein the peripheral opioid antagonist is not enterically coated.

99. The formulation of claim 87, wherein the formulation is constructed and arranged to release the peripheral opioid antagonist in the stomach, the small intestine, and the colon.

100. The formulation of claim 87, wherein the formulation is constructed and arranged to release the peripheral opioid antagonist in the small intestine and colon.

101. The formulation of claim 87, wherein the formulation is constructed and arranged to release the peripheral opioid antagonist in the small intestine.

102. The formulation of claim 87, wherein the formulation is constructed and arranged to release the peripheral opioid antagonist only in the colon.

103. The formulation of claim 87, wherein the formulation is constructed and arranged to release immediately substantially all of the peripheral opioid antagonist in the stomach.

104. The formulation of claim 80, wherein the peripheral opioid antagonist is in a sustained release material.

105. The formulation of claim 104, wherein the stool softener is not in a sustained release material.

106. The formulation of claim 80, wherein the stool softener is in a sustained release material.

107. The formulation of claim 106, wherein the peripheral opioid antagonist is not in a sustained release material.

108. The formulation of claim 80, wherein the peripheral opioid antagonist is a quaternary derivative of noroxymorphone.

109. The formulation of claim 108, wherein the peripheral opioid antagonist is methylnaltrexone.

110. The formulation of claim 109, wherein the methylnaltrexone is present in a range from 50 to 250 mg.

111. The formulation of claim 110, wherein the formulation further comprises an opioid.

112. A kit comprising:
a package containing a formulation of a peripheral opioid antagonist and a laxative and/or a stool softener.

113. (canceled)

114. The kit of claim 112, wherein the peripheral opioid antagonist is in a first container and the laxative and/or stool softener are in a container different from the first container.
